(19) United States
(12) Patent Application Publication
(10) Pub. No.: US 2005/0037030 A1
(43) Pub. Date: Feb. 17, 2005

(54) Stable topical formulation of clarithromycin

(76) Inventors: Mukesh Kumar, Punjab (IN); Ajay Kumar Singla, Chandigarh (IN); Vinod Kumar Arora, New Delhi (IN); Rajiv Malik, Wien (AT)

Correspondence Address: Jayadeep R Deshmukh;Ranbaxy Inc, 600 College Road East, Suite 2100, Princeton, NJ, 08540, US

(21) Appl. No.: 10/498,407

(22) PCT Filed: Dec. 12, 2002
(86) PCT No.: PCT/IB02/05323

(30) Foreign Application Priority Data
Dec. 13, 2001 (IN) 1343/DEL/01

(51) Int. Cl. A61K 31/7048; A61K 9/00

(52) U.S. Cl. 424/400; 514/28

(57) ABSTRACT

This invention relates to a stable topical formulation of clarithromycin and its use in the treatment of acne.

### FIELD OF THE INVENTION

This invention relates to a stable topical formulation of clarithromycin and its use in the treatment of acne.

### BACKGROUND OF THE INVENTION

Acne is a common inflammatory disease of the sebaceous glands. Corynebacterium acnes microorganism is typically responsible for the acne infections.

Various therapeutic methods that are currently used for treating acne include oral and topical bacteriostatics as well as systemic antibiotics. Tetracycline has been the traditional drug of choice, but other antibiotics such as erythromycin, lincomycin and clindamycin have also been prescribed for this use. While oral administration of these drugs often constitutes an effective treatment regimen for acne, it has disadvantages which include exposure of the entire body to the antibiotic as against localized lesions where it is required.

Topical application of antibiotics minimizes the antibiotic levels in the circulatory and gastrointestinal systems while killing the bacteria present in the lesions. This is a convenient, non-invasive route effective for the treatment of localized infections of the skin such as acne.

U.S. Pat. No. 4,132,781 describes compositions for the topical treatment of signs and symptoms of acne containing an antibiotic of the erythromycin family together with 2-pyrrolidone or an N-lower alkyl-2-pyrrolidone.

U.S. Pat. No. 4,469,684 describes pharmaceutical compositions containing zinc erythromycin and 50-99% of t-butanol for the treatment of acne.

U.S. Pat. No. 4,299,826 describes compositions for topical application in the treatment of skin disorders and dermatoses of bacterial origin comprising a minor proportion of an antibiotic agent selected from erythromycin and its derivatives and a carrier comprising penetration enhancing amount of diisopropyl sebacate and alcohol.

U.S. Pat. No. 5,455,037 describes stable topical cream compositions for treating dermal microbial infections in animals containing erythromycin, a polysiloxane, ethanol, water and an emulsifier.

Although these patents describe topical preparations of erythromycin and their salts and derivatives, none of them exemplify erythromycin derivatives like clarithromycin. Clarithromycin is a macrolide antibiotic which has strong antibacterial activity against gram-positive bacteria and is generally considered to have more potent antibacterial activity than the parent drug erythromycin.

The therapeutic efficacy of an antibiotic composition applied depends upon the ability of the applied composition to be carried through the epidermis and into the deeper layers of the skin as well as into follicles and sebum plugged follicles which contains causative microorganism for acne infections.

### SUMMARY OF THE INVENTION

It has now unexpectedly been discovered that clarithromycin may be effectively administered topically for the treatment of acne through the use of the formulations herein described. The preparation of a topical formulation of clarithromycin was a challenge due to its instability in aqueous media.

The invention relates to a stable topical formulation of clarithromycin comprising clarithromycin mixed in oil phase; a non-aqueous phase or emulsion in water; and other pharmaceutically acceptable excipients.

The invention also relates to a method of treating acne in humans or animals in need of such treatment comprising applying topically a formulation containing clarithromycin.

### DETAILED DESCRIPTION OF THE INVENTION

The amount of clarithromycin which may be used in the present invention varies depending upon the particular condition to be treated, severity of the condition, and other like factors. Accordingly, emulsion formulations of different strengths may be formulated containing clarithromycin ranging from about 0.1% to about 10% by weight of the formulation.

In accordance with the present invention, clarithromycin is mixed in a suitable oil phase such as monoglycerides, diglycerides, triglycerides, fatty acids, fatty alcohols, vegetable oils, mineral oils, their derivatives and mixture thereof. The amount of oil phase used depends upon the amount of clarithromycin that needs to be solubilized. The amount of oil phase which may be used in the present invention ranges from about 5% to about 40% by weight of the formulation. These solubilzers not only solubilize the drug, they also protect the drug from the surrounding aqueous environment and aid in better penetration of the drug though the skin.

The clarithromycin oil mix may either be emulsified in water or added to a non-aqueous vehicle. In a formulation where the clarithromycin oil mix is added to a non-aqueous vehicle, the non-aqueous vehicle is selected from high and low molecular weight polyethylene glycols, cetostearyl alcohol, cetyl alcohol, cetyl ester wax, lanolin alcohol, microcrystalline wax, non-ionic emulsifying wax, stearyl alcohol, white wax, paraffin, and the like.

The formulation may further contain other pharmaceutically acceptable excipients such as emulsion forming agents/emulsifiers, gelling agents, antioxidants, preservatives, neutralizing/pH modifying agents, and chelating agents.

The emulsion forming agents or emulsifier which may be used in these formulations include carbomers, polyoxyethylene castor oil derivatives, sorbitan fatty acid esters, polyoxyethylene sorbitan fatty acid esters, polyoxyethylene alkyl ethers,caprylic and capric triglycerides, polyethylene glycol esters and others belonging to the class of non-ionic surfactants. The amount of emulsion forming agents or emulsifier which may be used in the present invention ranges from about 5% to about 20% by weight of the formulation.

The gelling agents which may be used in these formulations include conventional gelling agents well known for their gelling properties, such as, for example, cellulose ethers such as hydroxypropyl cellulose, hydroxymethyl cellulose, hydroxypropyl methylcellulose, carboxymethyl cellulose, sodiumcarboxy methylcellulose, hydroxycellulose, and the like; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like, methacrylates such as those availale under the tradename Eudragit® from Rohm Pharma and polyacrylates such as those available under the brand name “Carbopol®” from B.F. Goodrich. Other gelling agents includes polyoxyethylene—polyoxypropylene copolymes (poloxamers) such as those available under the tradename “Lutrol®”, and the like.

The amount of gelling agent which may be used in the formulation would vary depending upon the gelling agent used. A low concentration of the gelling agent makes the formulation loose or fluid which runs on application, while a higher concentration would result in stiff formulations that are not easily spreadable. The gelling agents may be present from about 0.3% to about 40% by weight, for example, from about 0.5% to about 30% by weight of the formulation.

The chelating agents which may be used in these formulations include conventional chelating agents known in the art, such as, for example, disodium edetate. The amount of chelating agent which may be used ranges from about 0.005% to about 2.0% by weight of the formulation.

In accordance with the present invention, the formulation may also contain antioxidants from about 0.005% to about 1.0% by weight of the formulation, such as, for example, butylated hydroxy anisole (BHA), butylated hydroxy toluene (BHT), tertiary butyl hydroquinone, propyl gallate, α-tocopherol, and the like. The formulation may further contain preservatives from about 0.1% to about 2.0% by weight of the formulation. The preservatives which may be used include phenoxyethanol, methyl paraben, propyl paraben, butyl paraben, benzyl alcohol, and the like.

In accordance with the present invention, the formulation may also contain a pH modifying agent. The pH of the formulation in accordance with the present invention may be adjusted to between 4.0 and 8.0. Preferably, it may be adjusted between pH 6.0 and 7.0. The pH modifying agent may be selected from any well known and pharmacologically safe inorganic or organic basic salt. Examples of inorganic basic salts may include magnesium oxide, magnesium hydroxide, calcium hydroxide, sodium hydroxide, potassium hydroxide, potassium carbonate, sodium bicarbonate, and the like. Examples of organic basic salts may include methanolanine, ethanolamine, propanolamine, trimethanolamine, triethanolamine, alkylamines such as methylamine, ethylamine, butylamine, isopropylamine, and the like.

The formulations described herein have good stability, adhere well to skin and have a smooth feel. The formulations of this invention can be topically applied to affected areas of the skin in any convenient form, e.g. gel, cream, ointment, lotion, spray, etc.

The following examples illustrate preferred topical formulations prepared and used in the manner of this invention, but are not intended to be limiting thereof.

### EXAMPLES 1-5

Oil in Water Emulsion Formulations

| | Qty (% w/w) | | | | |
|---|---|---|---|---|---|
| Ingredients | 1 | 2 | 3 | 4 | 5 |
| Clarithromycin | 1.0 | 1.0 | 1.0 | 1.0 | 1.0 |
| Oleic acid | 5.0 | 5.0 | 5.0 | 5.0 | 5.0 |
| Isopropyl | 10.0 | 20.0 | 15.0 | 20.0 | 10.0 |
| myristate | | | | | |
| Phenoxyethanol | 1.0 | 1.0 | 1.0 | 1.0 | 1.0 |
| Tween 80 | 2.0 | 2.0 | 2.0 | 2.0 | 2.0 |
| Lutrol F127 | — | — | — | — | 15.0 |
| Carbopol 940 | 0.5 | 0.1 | 0.5 | 0.4 | — |
| Carbome 1342 | 0.6 | 0.6 | 0.6 | 0.6 | 0.6 |
| Triethanolamine | 1.0 | 1.25 | 1.0 | 1.25 | — |
| Disodium Edetate | 0.1 | 0.1 | 0.1 | 0.1 | 0.1 |
| Butylated | 0.05 | 0.05 | 0.05 | 0.05 | 0.05 |
| Hydroxy Anisole | | | | | |
| Sodium | 0.78 v/w | — | 0.76 v/w | 0.63 v/w | 0.7 v/w |
| Hydroxide | | | | | |
| (5% solution) | | | | | |
| to pH 7.0 w/v | | | | | |
| Fragrance | 0.39 | 0.39 | 0.39 | 0.33 | 0.39 |
| Purified Water | q.s. | q.s. | q.s. | q.s. | q.s. |

Butylated hydroxy anisole and clarithromycin were dissolved in oleic acid followed by the addition of isopropyl myristate, carbomer 1342, polysorbate 80 and a fraction of the purified water to form an emulsion. Gelling agent, such as carbopol 940 was separately dispersed in purified water and the dispersion formed was added to the clarithromycin emulsion with stirring. Fragrance was added. The formulation was neutralized by the addition of triethanolamine and the pH was adjusted to 7.0 with sodium hydroxide. The final weight was made up with purified water.

### EXAMPLE 6

Clarithromycin Lotion

| Ingredient | Qty (% w/w) |
|---|---|
| Clarithromycin | 1.00 |
| Butylated Hydroxy Anisole | 0.05 |
| Oleic acid | 5.0 |
| Isopropyl myristate | 15.00 |
| Pemulen TR2 (Carbomer 1342) | 0.60 |
| Phenoxy ethanol | 1.00 |
| Disodium edetate | 0.1 |
| Tween 80 | 2.0 |
| Triethanolamine | 0.60 |
| Fragrance | 1.0 |
| Pure Water | q.s. |

The process of making the lotion was similar to that used for making the emulsion.

### EXAMPLE -7

Ointment Formulation

| Ingredient | Qty (% w/w) |
|---|---|
| Clarithromycin | 1.00 |
| Oleic acid | 5.00 |
| Butylated Hydroxy Anisole | 0.05 |
| PEG 4000 | 25 |
| Fragrance 00329 | 0.8 |
| PEG 400 | qs |

PEG 4000 was melted by heating it to 70° C. followed by the addition of a portion of PEG 400. Clarithromycin was separately mixed with BHA and oleic acid and the fragrance. The clarithromycin mix was added to be melted PEG and mix. The weight was made with PEG 400.

The formulation as described in Example 3 was subjected to an open, non-comparative study on seventy patients in the age group of 13 to 31 years and a male, female ratio of 1:1. The patients were treated with topical application of clarithromycin 1% gel twice a day for six weeks.

The efficacy study was monitored for the following:
(i) number of inflammatory (papules/pustules and nodules) and non-inflammatory (comedones) lesions.
(ii) Scores for facial oiliness, erythema, scaling, burning and pruritis recorded on a scale of 0 to 3 (none to severe)
(iii) Physician and patients assessed global improvement on a scale of 0 to 6 (worsened to complete clearing) and
(iv) Patient assessed cosmetic acceptability on a scale of 0 to 5 (unfavaourable to highly favourable).

Transient adverse events, mild in nature were reported by 6 patients (8.6%). No serious event was reported indicating that clarithromycin gel was well tolerated.

After evaluating all the parameters the physicians and patients gave their assessment of the global improvement in the patients conditions as given in the following Table.

Global Improvement—Physician Assessment

| | No (%) |
|---|---|
| | of patients (n = 66) |
| Complete clearing: No sign or | 2 (3.0%) |
| symptoms of disease | |
| Excellent response 75-99% | 35 (53.0%) |
| improvement | |
| Good response: 50-74% | 18 (27.3%) |
| improvement | |
| Fair response: 25-49% improvement | 8 (12.1%) |
| Poor response: 1-24% improvement | 2 (3.0%) |
| Condition unchanged | 1 (1.5%) |
| Conditioned worsened | 0.00 |

As can be seen from the data complete clearing was observed in 2 patients (3%) and good to excellent response in 53 patients (80.3%) indicating the effectiveness of the treatment.

Global Improvement—Patient Assessment

| | No (%) |
|---|---|
| | of patients (n = 66) |
| Complete clearing: No sign or | 4 (6.1%) |
| symptoms of disease | |
| Excellent response 75-99% | 38 (57.6%) |
| improvement | |
| Good response: 50-74% | 13 (19.7%) |
| improvement | |
| Fair response: 25-49% improvement | 8 (12.1%) |
| Poor response: 1-24% improvement | 3 (4.6%) |
| Condition unchanged | 0.00 |
| Conditioned worsened | 0.00 |

Good to excellent response and complete clearing was documented in 55 patients (83%) and less than 50% improvement was noted in only 11 patients (16.7%). None of the patients assessed their conditions as unchanged or worsened. This once again showed the efficacy of the treatment.

Toxicity Studies

Subchronic dermal toxicity was conducted on Sprague Dawley Rats. 0-1000 mg/kg body weight of clarithromycin formulation was applied on the shorn back of animals daily for 28 days. No abnormalities were reported in the 28 days application period nor in the 14 days recovery period indicating the safety of the formulation.

While the present invention has been described in terms of its specific embodiments, certain modifications and equivalents will be apparent to those skilled in the art and are intended to be included within the scope of the present invention.

1. A stable topical formulation of clarithromycin comprising clarithromycin mixed in oil phase; a non-aqueous phase or emulsion in water; and other pharmaceutically acceptable excipients.

2. The formulation of claim 1 wherein the clarithromycin comprises from about 0.1% to about 10% by weight of said formulation.

3. The formulation of claim 1 wherein the oil phase is selected from the group consisting of monoglycerides, diglycerides, triglycerides, fatty acids, fatty alcohols, vegetable oils, mineral oils and derivatives thereof.

4. The formulation of claim 3 wherein the fatty acids are selected from the group consisting of lauric acid, myristic acid, palmitic acid, stearic acid, oleic acid, linoleic acid, linolenic acid, arachidonic acid and derivatives thereof.

5. The formulation of claim 3 wherein the vegetable oils are selected from the group consisting of soyabean oil, safflower oil, corn oil, cotton seed oil, peanut oil, olive oil, coconut oil, linseed oil and mixtures thereof.

6. The formulation of claim 3 wherein the mineral oil is liquid paraffin.

7. The formulation of claim 1 wherein the oil phase comprises from about 2% to about 40% by weight of said formulation.

8. The formulation of claim 1 wherein the formulation is an oil in water emulsion.

9. The formulation of claim 1 wherein the non-aqueous phase is selected from the group consisting of high and low molecular weight polyethylene glycols, cetostearyl alcohol, cetyl alcohol, cetyl ester wax, lanolin alcohol, lanolin, microcrystalline wax, nonionic emulsifying wax, stearyl alcohol, white wax, petrolatum, paraffin and mixtures thereof.

10. The formulation of claim 1 wherein the pharmaceutically acceptable excipients comprises emulsifying agents, gelling agents, chelating agents, pH-modifying agents, preservatives and antioxidants.

11. The formulation of claim 10 wherein the emulsifying agents are selected from the group consisting of carbomers, polyoxyethylene castor oil derivatives, sorbitan fatty acid esters, polyoxyethylene sorbitan fatty acid esters, polyoxyethylene alkyl ethers, caprylic and capric triglycerides, polyethyleneglycol esters and others belonging to the class of non-ionic surfactants.

12. The formulation of claim 10 wherein the emulsifying agent comprises from about 5% to about 20% by weight of said formulation.

13. The formulation of claim 10 wherein the gelling agents are selected from the group consisting of cellulose ethers, vinyl alcohols, vinyl pyrrolidones, gums, methacrylates, polyacrylates, and poloxomers.

14. The formulation of claim 13 wherein the cellulose ethers are selected from the group consisting of hydroxypropyl cellulose, hydroxymethyl cellulose, hydroxypropyl methylcellulose, carboxymethyl cellulose, sodium carboxymethyl cellulose, and hydroxycellulose.

15. The formulation of claim 13 wherein the gums are selected from the group consisting of karaya gum, locust bean gum, guar gum, gelan gum, xanthan gum, gum arabic, tragacanth gum, carrageenan gum, pectin, agar, alginic acid, and sodium alginate.

16. The formulation of claim 13 wherein the methacrylates are those sold under the trade name of Eudragit® from Rohm Pharma.

17. The formulation of claim 13 wherein the polyacrylates are those available under the trade name “Carbopol®” from B. F. Goodrich.

18. The formulation of claim 10 wherein the gelling agents comprises from about 0.3% to about 40% by weight of said formulation.

19. The formulation of claim 18 wherein the gelling agents comprises from about 0.5% to about 30% by weight of said formulation.

20. The formulation of claim 10 wherein the chelating agent is disodium edetate.

21. The formulation of claim 10 wherein the chelating agent comprises from about 0.005% to about 2.0% by weight of said formulation.

22. The formulation of claim 10 wherein the pH modifying agent is selected from the group consisting of magnesium oxide, magnesium hydroxide, calcium hydroxide, sodium hydroxide, potassium hydroxide, potassium carbonate, sodium carbonate and the like and organic salts such as methanolamine, ethanolamine, propanolamine, ethylamine, butylamine, and isopropylamine.

23. The formulation of claim 22 wherein the pH modifying agent is triethanolamine.

24. The formulation of claim 10 wherein the preservatives are selected from the group consisting of phenoxyethanol, benzyl alcohol, methyl paraben, propyl paraben, and butyl paraben.

25. The formulation of claim 10 wherein the preservatives comprises from about 0.1% to about 2% by weight of said formulation.

26. The formulation of claim 10 wherein the antioxidants are selected from the group consisting of butylated hydroxyanisole, butylated hydroxytoluene, propylgallate, a-tocopherol, and tertiary butyl hydroquinone.

27. The formulation of claim 10 wherein the antioxidant comprises from about 0.005% to about 1.0% by weight of said formulation.

28. The formulation of claim 1 may be adjusted to a pH of from about 4.0 to about 8.0.

29. The formulation of claim 28 may be adjusted to a pH of from about 6.0 to about 7.0.

30. The formulation of claim 1 wherein the topical formulation is a gel, cream, ointment, lotion, or spray.

31. The formulation of claim 30 wherein the topical formulation is an ointment.

32. A method of treating acne in humans or animals in need of such treatment, comprising applying topically a formulation according to claim 1.

33. A method of treating acne in humans or animals in need of such treatment, comprising applying topically a formulation containing clarithromycin.
